(19) United States
(12) Patent Application Publication
(10) Pub. No.: US 2024/0215917 A1
(43) Pub. Date: Jul. 4, 2024

(54) Optical and Electrical Diagnostic Systems and Methods Thereof

(71) Applicant: Bard Access Systems, Inc., Salt Lake City, UT (US)

(72) Inventors: Steffan Sowards, Salt Lake City, UT (US); Anthony K. Misener, Bountiful, UT (US); William Robert McLaughlin, Bountiful, UT (US)

(21) Appl. No.: 18/607,165

(22) Filed: Mar. 15, 2024

Related U.S. Application Data
(62) Division of application No. 17/217,852, filed on Mar. 30, 2021, now Pat. No. 11,931,179.
(60) Provisional application No. 63/002,041, filed on Mar. 30, 2020.

(51) Int. Cl. A61B 5/00; A61B 5/107; A61M 25/00; A61B 34/20

(52) U.S. Cl. CPC A61B 5/6852; A61B 5/1076; A61M 25/0067; A61B 2034/2061

(57) ABSTRACT

Optical-and-electrical medical systems and methods thereof are disclosed. Such a medical system can include a console, an optical shape-sensing (“OSS”) medical device including an optical-fiber stylet, one or more electrical medical devices, and a relay module for establishing one or more optical or electrical relay connections between the relay module and a remainder of the medical system including the console. The console can be configured for converting reflected optical signals from the optical-fiber stylet into shapes thereof for display. The relay module can include a primary receptacle configured to accept insertion of a medical barrier-piercing plug of the OSS medical device and establish a through-barrier optical connection therebetween. The relay module also can include one or more secondary receptacles configured to accept insertion of a corresponding number of electrical plugs of the one-or-more electrical medical devices and establish one or more under-barrier electrical connections therebetween.

This application is a division of U.S. patent application Ser. No. 17/217,852, filed Mar. 30, 2021, now U.S. Pat. No. 11,931,179, which claims the benefit of priority to U.S. Provisional Application No. 63/002,041, filed Mar. 30, 2020, each of which is incorporated by reference in its entirety into this application.

### BACKGROUND

At times, a tip of a peripherally inserted central catheter (“PICC”) or central venous catheter (“CVC”) can move becoming displaced from an ideal position in a patient's superior vena cava (“SVC”). A clinician believing such a PICC or CVC has displaced typically checks for displacement by chest X-ray and replaces the PICC or CVC if necessary. Because X-rays expose patients to ionizing radiation, medical devices such as PICCs and CVCs are being developed with integrated optical-fiber stylets for clinicians to check easily and safely for displacement thereof. However, in order for the clinicians to check for displacement, the PICCs or CVCs, which are sterile as provided, need to be at least optically connected to non-sterile capital equipment without compromising sterile conditions. Therefore, there is a need for an optical medical system that allows for single-use medical devices such as the foregoing PICCs and CVCs to be at least optically connected to non-sterile capital equipment without compromising sterile conditions. Disclosed herein are optical and electrical medical systems, and methods thereof.

### SUMMARY

Disclosed herein is a relay module including, in some embodiments, a housing, a primary receptacle disposed in the housing, one or more secondary receptacles disposed in the housing, and one or more cables extending from the housing. The primary receptacle is configured to accept insertion of a barrier-piercing plug associated with an optical shape-sensing (“OSS”) medical device and establish a through-barrier connection between the relay module and the OSS medical device. The through-barrier connection is selected from a through-barrier optical connection and a through-barrier optical-and-electrical connection. The one-or-more secondary receptacles are configured to accept insertion of a corresponding number of electrical plugs associated with one or more electrical medical devices and establish a corresponding number of under-barrier electrical connections between the relay module and the one-or-more electrical medical devices. The one-or-more cables include a corresponding number of relay-module plugs for establishing one or more relay connections between the relay module and a remainder of a medical system configured to receive optical and electrical signals from the relay module. The one-or-more relay connections are selected from a relay optical connection, a relay optical-and-electrical connection, and a relay electrical connection, provided at least the relay optical connection or the relay optical-and-electrical connection is selected.

In some embodiments, the relay module further includes an optical fiber having one or more optical-fiber cores extending from the primary receptacle through a primary cable of the one-or-more cables to a corresponding primary plug of the number of relay-module plugs. The optical fiber is configured to convey input optical signals from an optical interrogator of the medical system to an optical-fiber stylet of the OSS medical device once the through-barrier connection is established. The optical fiber is also configured to convey reflected optical signals from the optical-fiber stylet to the optical interrogator when the through-barrier connection is established.

In some embodiments, the relay module further includes one or more electrical leads extending from a number of receptacles including the primary receptacle and the one-or-more secondary receptacles through any cable of the one-or-more cables to the corresponding relay-module plug thereof. The one-or-more electrical leads are configured to convey electrical signals from the relay module to the medical system.

In some embodiments, the relay module further includes one or more sensors selected from a gyroscope, an accelerometer, and a magnetometer disposed within the housing.

The one-or-more sensors are configured to provide sensor data for determining a reference plane for shape sensing with the OSS medical device.

In some embodiments, the relay module of further includes a light-emitting diode (“LED”) disposed in the housing proximate the primary receptacle. The LED is configured to illuminate upon establishing the relay optical-and-electrical connection or any relay electrical connection of the number of relay electrical connections for finding the primary receptacle when the relay module is under a medical barrier.

In some embodiments, the relay module is amenable to high-level disinfection or sterilization.

In some embodiments, the housing includes a patient-facing surface configured to be adhered to a patient, thereby enabling the relay module to be secured to the patient.

Disclosed herein is a medical system including, in some embodiments, a console, an OSS medical device configured for shape sensing, one or more electrical medical devices, and a relay module. The console includes memory and one or more processors for converting reflected optical signals from an optical-fiber stylet into shapes thereof for display. The console further includes an integrated optical interrogator for converting the reflected optical signals into the shapes of the optical-fiber stylet. Alternatively, the console is communicatively coupled to a stand-alone optical interrogator configured for converting the reflected optical signals into the shapes of the optical-fiber stylet. The OSS medical device includes the optical-fiber stylet and a barrier-piercing plug. Each electrical medical device of the one-or-more electrical medical devices includes at least one electrical plug. The relay module includes a housing, a primary receptacle disposed in the housing, one or more secondary receptacles disposed in the housing, and one or more cables extending from the housing. The primary receptacle is configured to accept insertion of the barrier-piercing plug associated with the OSS medical device and establish a through-barrier connection between the relay module and the OSS medical device. The through-barrier connection is selected from a through-barrier optical connection and a through-barrier optical-and-electrical connection. The one-or-more secondary receptacles are configured to accept insertion of the at least one electrical plug of each electrical medical device of the one-or-more electrical medical devices and establish a corresponding number of under-barrier electrical connections between the relay module and the one-or-more electrical medical devices. The one-or-more cables include a corresponding number of relay-module plugs for establishing one or more relay connections between the relay module and either the console or a combination of the console and the stand-alone optical interrogator. The one-or-more relay connections are selected from a relay optical connection, a relay optical-and-electrical connection, and a relay electrical connection, provided at least the relay optical connection or the relay optical-and-electrical connection is selected.

In some embodiments, the relay module further includes an optical fiber having one or more optical-fiber cores extending from the primary receptacle through a primary cable of the one-or-more cables to a corresponding primary plug of the number of relay-module plugs. The optical fiber is configured to convey input optical signals from either the integrated optical interrogator or the stand-alone optical interrogator to the optical-fiber stylet of the OSS medical device once the through-barrier connection is established. The optical fiber is also configured to convey reflected optical signals from the optical-fiber stylet to the integrated optical interrogator or the stand-alone optical interrogator when the through-barrier connection is established.

In some embodiments, the relay module further includes one or more electrical leads extending from a number of receptacles including the primary receptacle and the one-or-more secondary receptacles through any cable of the one-or-more cables to the corresponding relay-module plug thereof. The one-or-more electrical leads are configured to convey electrical signals from the relay module to the console.

In some embodiments, the relay module further includes one or more relay module-based sensors selected from a gyroscope, an accelerometer, and a magnetometer disposed within the housing. The one-or-more relay module-based sensors are configured to provide relay module-based sensor data for determining a reference plane for shape sensing with the OSS medical device.

In some embodiments, the relay module of further includes an LED disposed in the housing proximate the primary receptacle. The LED is configured to illuminate upon establishing an electrical connection of the one-or-more relay connections for finding the primary receptacle when the relay module is under a medical barrier.

In some embodiments, the relay module is amenable to high-level disinfection or sterilization.

In some embodiments, the housing includes a patient-facing surface configured to be adhered to a patient, thereby enabling the relay module to be secured to the patient.

In some embodiments, the one-or-more electrical medical devices include one or more electrocardiogram (“ECG”) patches.

In some embodiments, the one-or-more electrical medical devices include an arm band. The arm band has one or more arm band-based sensors selected from an arm band- based gyroscope, accelerometer, and magnetometer. The one-or-more arm band-based sensors are configured to provide arm band-based sensor data for reference-plane determination for the shape sensing with the OSS medical device.

Also disclosed herein is a method of a medical system. The method includes, in some embodiments, a relay-module placing step, an electrical-plug inserting step, a medical-drape placing step, and a barrier-piercing-plug inserting step. The relay-module placing step includes placing a relay module on a surface. The electrical-plug inserting step includes inserting one or more electrical plugs corresponding to one or more electrical medical devices into a corresponding number of secondary receptacles of the relay module. The electrical-plug inserting step establishes one or more under-barrier electrical connections between the one-or-more electrical devices and the relay module. The medical-drape placing step includes placing a medical barrier over the relay module. The medical-drape placing step establishes a sterile field over the relay module. The barrier-piercing-plug inserting step includes inserting a barrier-piercing plug of an OSS medical device through the medical barrier and into a primary receptacle of the relay module. The barrier-piercing-plug inserting step establishes a through-barrier optical connection or a through-barrier optical-and-electrical connection between the OSS medical device in a sterile field and the relay module in a non-sterile field.

In some embodiments, the relay-module placing step includes placing the relay module on a chest of a patient. The relay-module placing step establishes a reference plane for shape sensing with the OSS medical device.

In some embodiments, the method further includes an identifying step and a palpating step. The identifying step includes identifying an illuminated LED disposed in a housing of the relay module proximate the primary receptacle. The palpating step includes palpating the primary receptacle under the medical barrier. Both the identifying step and the palpating step occur before the barrier-piercing-plug inserting step.

In some embodiments, the method further includes an establishing step of establishing one or more relay connections between the relay module and a remainder of the medical system for relaying optical and electrical signals from the relay module. The one-or-more relay connections are selected from a relay optical connection, a relay optical-and-electrical connection, and a relay electrical connection, provided at least the relay optical connection or the relay optical-and-electrical connection is selected.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 is a block diagram of a first medical system in accordance with some embodiments.

FIG. 2 is a block diagram of a second medical system in accordance with some embodiments.

FIG. 3 illustrates the second medical system in accordance with some embodiments.

FIG. 4 illustrates a cross-section of a catheter tube of an OSS medical device in accordance with some embodiments.

FIG. 5 illustrates a barrier-piercing plug of an extension tube of the OSS medical device for establishing an optical or optical-and-electrical connection in accordance with some embodiments.

FIG. 6 illustrates a detailed view of a relay module with a primary receptacle for establishing optical or optical-and-electrical connections in accordance with some embodiments.

FIG. 7 illustrates establishing a through-barrier optical connection or a through-barrier optical-and-electrical connection between the OSS medical device and the relay module in accordance with some embodiments.

FIG. 8 illustrates the second medical system in use during a patient procedure in accordance with some embodiments.

FIG. 9 illustrates the second medical system in use during a patient procedure with a medical barrier in accordance with some embodiments.

FIG. 10 illustrates an optical connector of the extension tube of the OSS medical device in accordance with some embodiments.

FIG. 11 illustrates an alternative relay module with an optical connector for establishing optical connections across a medical barrier in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

Unless defined otherwise, all technical and scientific terms used herein have the same meaning as commonly understood by those of ordinary skill in the art.

As set forth above, there is a need for an optical medical system that allows for single-use medical devices such as the foregoing PICCs and CVCs to be at least optically connected to non-sterile capital equipment without compromising sterile conditions. Disclosed herein are optical and electrical medical systems and methods thereof.

Features of the optical and electrical medical systems (“medical systems”) provided herein will become more apparent with reference to the accompanying drawings and the following description, which provide particular embodiments of the medical systems in greater detail. For context, the medical systems are described first followed by medical devices and relay modules of the medical systems, as well as methods of the foregoing.

### Medical Systems

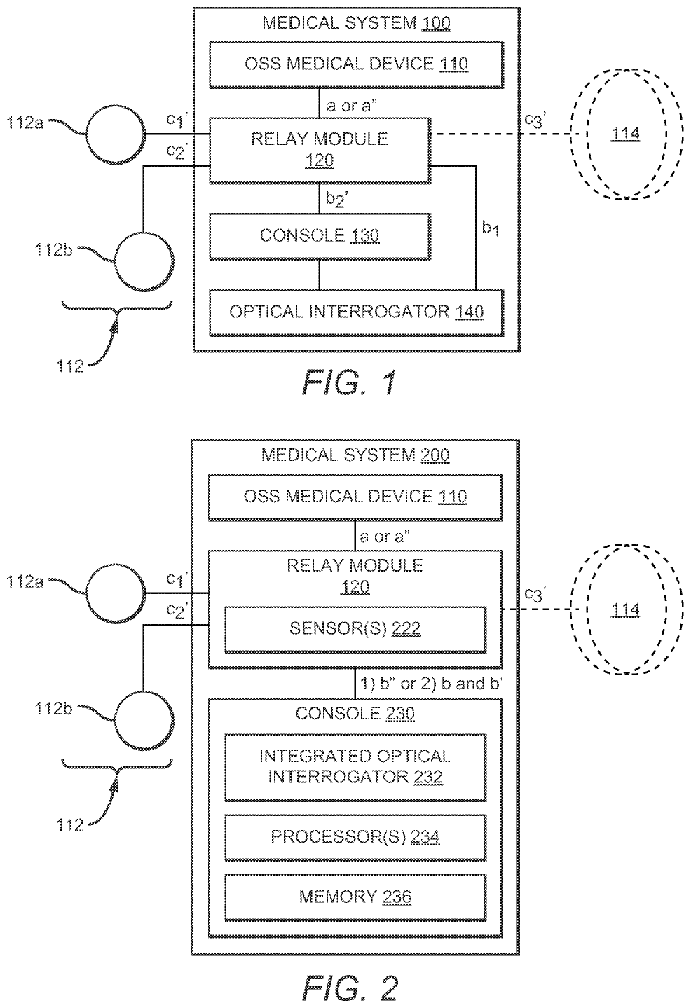

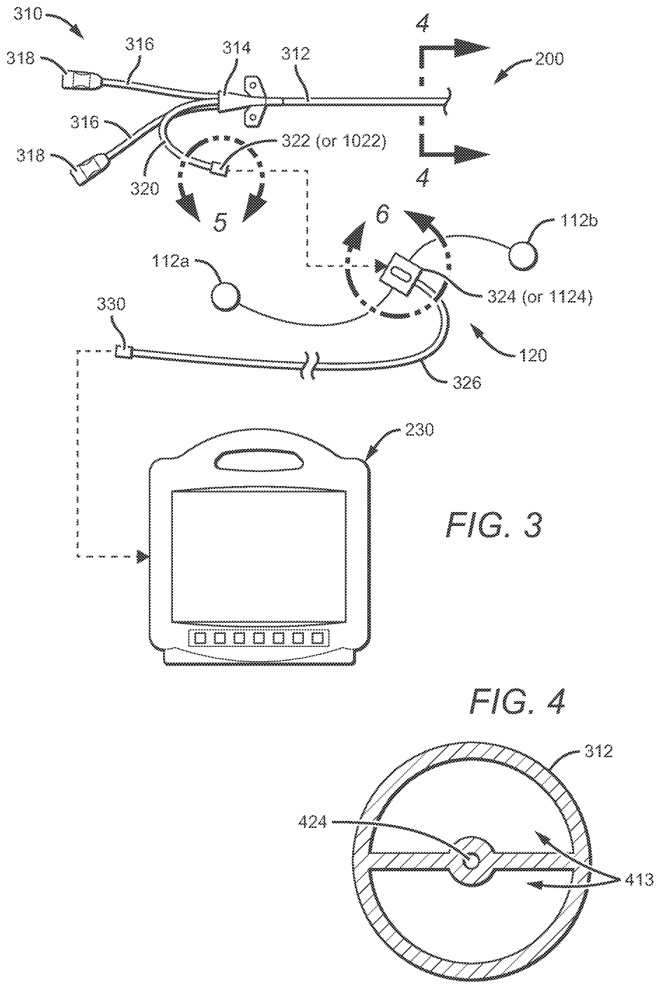

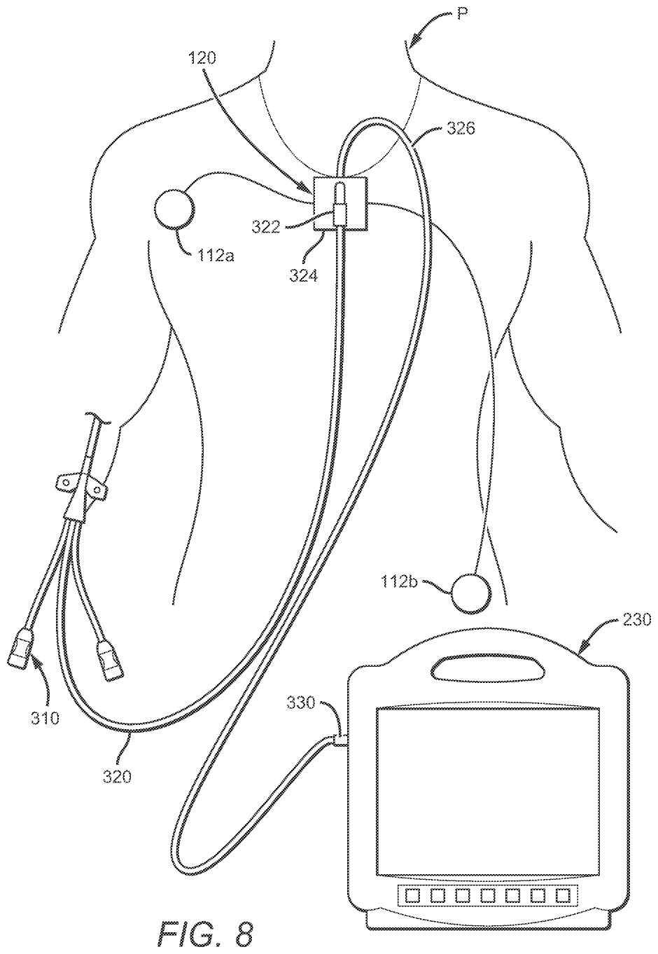

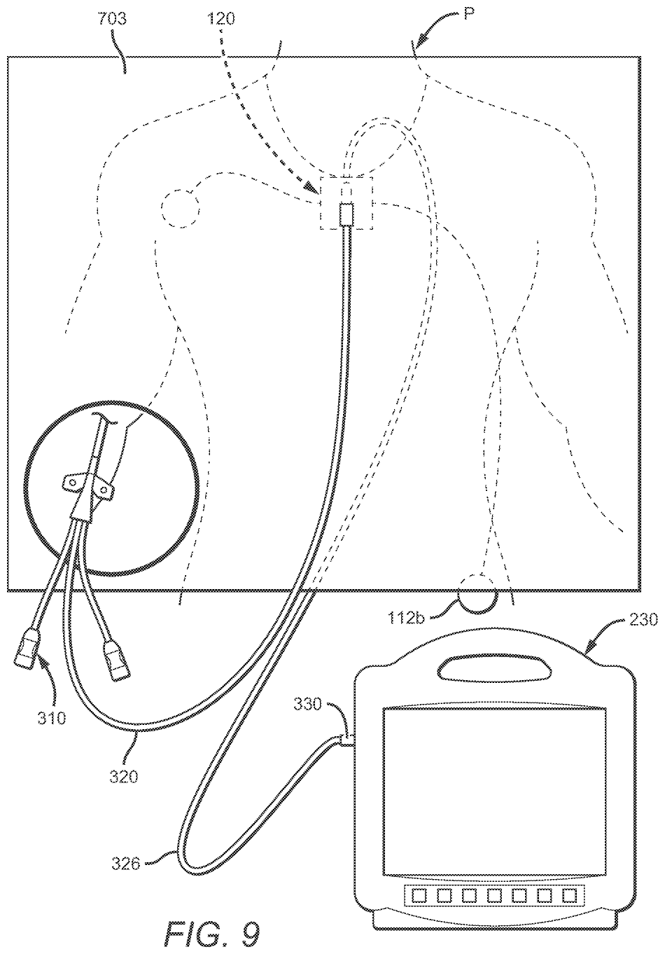

FIG. 1 is a block diagram of a first medical system 100 in accordance with some embodiments. FIG. 2 is a block diagram of a second medical system 200 in accordance with some embodiments. FIG. 3 illustrates the second medical system 200 in accordance with some embodiments. FIG. 8 illustrates the second medical system 200 in use during a patient procedure in accordance with some embodiments. FIG. 9 illustrates the second medical system 200 in use during a patient procedure with a medical barrier 703 in accordance with some embodiments.

As shown, the medical system 100 or 200 includes an OSS medical device 110, one or more electrical medical devices, a console 130 or 230, and a relay module 120 configured for connecting the OSS medical device 110 and the one-or-more electrical medical devices to a remainder of the medical system 100 or 200 such as the console 230. The OSS medical device 110 is typically used in a sterile field while the one-or-more electrical medical devices, the relay module 120, and the console 130 or 230 are typically used in a non-sterile field as defined by at least the medical barrier 703 (e.g., sterile drape).

The OSS medical device 110 includes at least an integrated optical-fiber stylet and a barrier-piercing plug. The optical-fiber stylet is a probe including one or more optical-fiber cores, wherein each core of the one-or-more optical-fiber cores has a number of fiber Bragg grating (“FBG”) sensors along a length thereof for shape sensing with the medical system 100 or 200. (See integrated optical-fiber stylet 424 in FIG. 4 for an example of the optical-fiber stylet of the OSS medical device 110.) The barrier-piercing plug is configured to establish at least a through-barrier optical connection between the OSS medical device 110 and the relay module 120. (See barrier-piercing plug 322 in FIG. 3 for an example of the barrier-piercing plug of the OSS medical device 110.) However, the OSS medical device 110 can also include electrical componentry such as an electrocardiogram (“ECG”) stylet and one or more electrical leads in support of the ECG stylet. As such, the barrier-piercing plug can be configured to establish a through-barrier optical-and-electrical connection between the OSS medical device 110 and the relay module 120 in some embodiments of the OSS medical device 110.

Certain features of the OSS medical device 110 are set forth in more detail below with respect to particular embodiments of the OSS medical device 110 such as the PICC 310. That said, some features (e.g., the optical fiber stylet, the ECG stylet, etc.) set forth below with respect to one or more embodiments of the OSS medical device 110 such as the PICC 310 can be shared among two or more embodiments of the OSS medical device 110. As such, “OSS medical device 110” is used herein to generically refer to more than one embodiment of the OSS medical device 110 when needed for expository expediency. This is despite certain features having been described with respect to particular embodiments of the OSS medical device 110 such as the PICC 310.

The one-or-more electrical medical devices can include, but are not limited to, electrocardiogram (“ECG”) patches 112 (e.g., an ECG patch 112a configured as a right-arm ECG patch, an ECG patch 112b configured as a left-leg ECG patch, . . . , and an ECG patch 112n), a companion arm band 114, or both the ECG patches 112 and the arm band 114. Each electrical medical device of the one-or-more electrical medical devices includes at least one electrical plug configured to establish an electrical connection between the electrical device thereof and the relay module 120.

The arm band 114 can include one or more sensors selected from at least a gyroscope, an accelerometer, and a magnetometer disposed within the arm band 114. The one-or-more sensors are configured to provide sensor data to the console 130 or 230 by way of the relay module 120 for determining a reference plane for shape sensing with the optical-fiber stylet of the OSS medical device 110.

While only shown for the console 230, each console of the consoles 130 and 230 includes memory 236 and one or more processors 234 configured for at least converting reflected optical signals from the optical-fiber stylet of the OSS medical device 110 into displayable shapes for the OSS medical device 110. The displayable shapes for the OSS medical device 110 can be displayed on an integrated display screen integrated into the console 130 or 230 or a display screen of a stand-alone monitor coupled to the console 130 or 230.

Each console of the consoles 130 and 230 can be further configured for converting electrical signals from the one-or-more electrical devices into displayable information for the one-or-more electrical devices. For example, for the optional ECG stylet or any ECG patch of the ECG patches 112, the console 130 or 230 can be configured to convert the electrical signals therefrom into an ECG trace for display. Such ECG traces can be useful in combination with the displayable shapes for the OSS medical device 110 for determining if the OSS medical device 110 has been displaced from its ideal position.

The medical system 100 further includes a stand-alone optical interrogator 140 communicatively coupled to the console 130, whereas the medical system 200 further includes an integrated optical interrogator 232 integrated into the console 230. The optical interrogator 140 or 232 is configured to send input optical signals into the optical-fiber stylet of the OSS medical device 110 by way of the relay module 120 and receive reflected optical signals from the optical-fiber stylet by way of the relay module 120. The optical interrogator 140 or 232 can be further configured to provide data to the console 130 or 230 corresponding to the reflected optical signals received for converting the reflected optical signals from the optical-fiber stylet of the OSS medical device 110 into the displayable shapes for the OSS medical device 110.

The relay module 120 includes a housing 324, a cable 326 extending from the housing 324, one or more optical-fiber cores 628 (“optical fiber 628”) extending through the housing 324 and along the cable 326, and one or more electrical leads extending through the housing 324 and along the cable 326 or another cable. (See FIG. 6 for the optical fiber 628.)

The relay module 120 is configured to establish at least an optical connection (e.g., a through-barrier optical connection) between the optical-fiber stylet of the OSS medical device 110 and the optical fiber 628 of the relay module 120. (See connections a and a″ in FIGS. 1 and 2, each connection of which includes at least an optical connection.) The relay module 120 is also configured with a plug 330 at a terminus of the cable 326 to establish at least another optical connection but between the optical fiber 628 of the relay module 120 and the stand-alone optical interrogator 140 or the console 230 including the integrated optical interrogator 232. (See connection b₁ in FIG. 1 and connection b or b″ in FIG. 2, each connection of which includes at least an optical connection.) The optical fiber 628 of the relay module 120 is configured to convey the input optical signals from the optical interrogator 140 or 232 to the optical-fiber stylet of the OSS medical device 110 and the reflected optical signals from the optical-fiber stylet to the optical interrogator 140 or 232.

The relay module 120 can also be configured to establish an electrical connection (e.g., a through-barrier electrical connection) between any electrical componentry of the OSS medical device 110 and the one-or-more electrical leads of the relay module 120, which electrical connection can be simultaneously established with the optical connection in an electrical-and-optical connection. (See connections a″ in FIGS. 1 and 2, each connection of which includes both an electrical-and-optical connection.) The plug 330 at the terminus of the cable 326 can be configured to establish another electrical connection but between the one-or-more electrical leads of the relay module 120 and the console 230 (i.e., the console 230 including the integrated optical interrogator 232). Such an electrical connection can be simultaneously established with the optical connection to the console 230 in another electrical-and-optical connection. (See connection b″ in FIG. 2, which includes both an electrical-and-optical connection.) Alternatively, another plug at a terminus of another cable of the relay module 120 can be configured to establish the other electrical connection between the one-or-more electrical leads of the relay module 120 and the console 130 or 230. (See connection b₂′ in FIG. 1 and b′ in FIG. 2, each connection of which includes at least an electrical connection.) The one-or-more electrical leads of the relay module 120 are configured to convey electrical signals from any electrical componentry of the OSS medical device 110 to the console 130 or 230.

Exclusive of the electrical connection between any electrical componentry of the OSS medical device 110 and the one-or-more electrical leads of the relay module 120, the relay module 120 can be configured to establish an electrical connection (e.g., an under-barrier electrical connection) between any electrical medical device of the one-or-more electrical medical devices (e.g., any ECG patch of the ECG patches 112, the arm band 114, etc.) and the one-or-more electrical leads of the relay module 120. (See connections c₁′, c₂′, and c₃′ in FIGS. 1 and 2, each connection of which includes at least an electrical connection.) As set forth above, the plug 330 at the terminus of the cable 326 can be configured to establish the other electrical connection between the one-or-more electrical leads of the relay module 120 and the console 230 (i.e., the console 230 including the integrated optical interrogator 232). Such an electrical connection can be simultaneously established with the optical connection to the console 230 in the other electrical-and-optical connection. (See connection b″ in FIG. 2, which includes both an electrical-and-optical connection.) Alternatively, the other plug at the terminus of the other cable of the relay module 120 can be configured to establish the other electrical connection between the one-or-more electrical leads of the relay module 120 and the console 130 or 230. (See connection b ′ in FIG. 1 and b′ in FIG. 2, each connection of which includes at least an electrical connection.) The one-or-more electrical leads of the relay module 120 are configured to convey electrical signals from the one-or-more electrical devices to the console 130 or 230.

It should be understood from the foregoing that a-type connections or through-barrier connections are between the OSS medical device 110 and the relay module 120, b-type connections or relay connections are between the relay module 120 and a remainder of the medical system 100 or 200 (e.g., a combination of the console 130 and the stand-alone optical interrogator 140 or the console 230 with the integrated optical interrogator 232), and c-type connections or under-barrier connections are between the one-or-more electrical medical devices and the relay module 120. A subscripted number associated with any type of connection of the a-, b-, and c-type connections numerically identifies a particular connection of a number of the a-, b-, or c-type connections. Lastly, an a-, b-, or c-type connection without a prime (“′”) or double prime (“″”) indicates an optical connection, an a-, b-, or c-type connection with a prime (e.g., “a′”) indicates an electrical connection, and an a-, b-, or c-type connection with a double prime (e.g., “a″”) indicates an optical-and-electrical connection.

The relay module 120 can further include one or more sensors 222 selected from at least a gyroscope, an accelerometer, and a magnetometer disposed within the housing 324. The one-or-more sensors 222 are configured to provide sensor data to the console 130 or 230 by way of the one-or-more electrical leads within the housing 324 and the cable 326 (or the other cable set forth above with respect to connection b₂′ in FIG. 1 and b′ in FIG. 2) for determining a reference plane for shape sensing with the optical-fiber stylet of the OSS medical device 110.

Certain features of the relay module 120 are set forth in more detail below with respect to particular embodiments of the relay module 120. That said, some features set forth below with respect to one or more embodiments of the relay module 120 are shared among two or more embodiments of the relay module 120 such as the relay module 1120. As such, “relay module 120” is used herein to generically refer to more than one embodiment of the relay module 120 when needed for expository expediency. This is despite certain features having been described with respect to particular embodiments of the relay module 120.

### Medical Devices

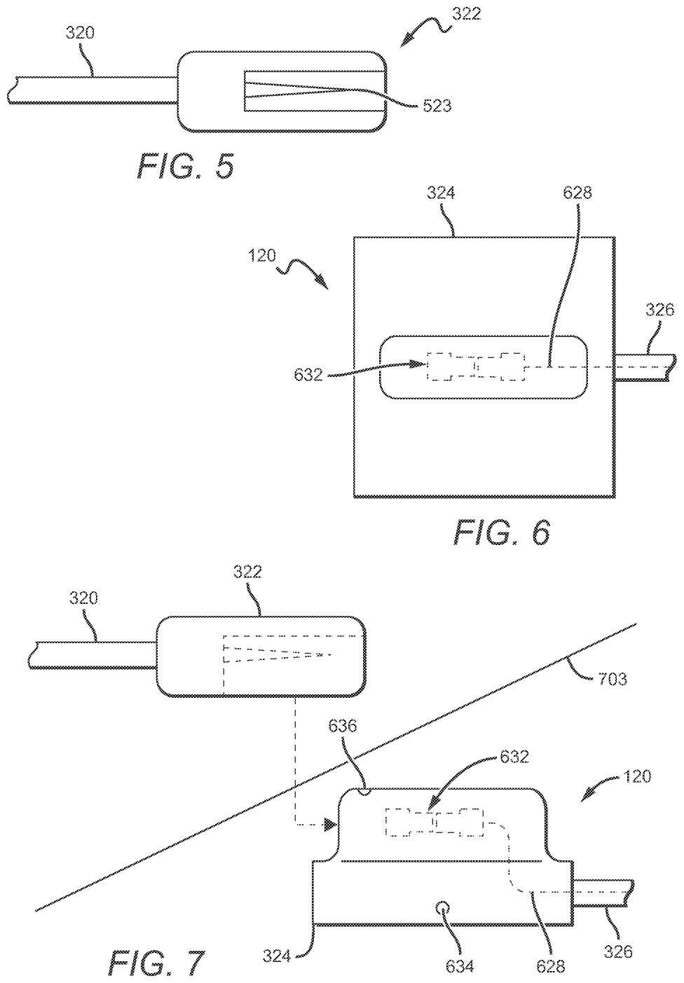

FIG. 3 also illustrates a PICC 310 as the OSS medical device 110 in accordance with some embodiments. FIG. 4 illustrates a cross-section of a catheter tube 312 of the PICC 310 including an integrated optical-fiber stylet 424 in accordance with some embodiments. FIG. 5 illustrates a barrier-piercing plug 322 of an extension tube or cable 320 of the PICC 310 for establishing an optical or optical-and-electrical connection in accordance with some embodiments.

As shown, the PICC 310 includes the catheter tube 312, a bifurcated hub 314, two extension legs 316, and two Luer connectors 318 operably connected in the foregoing order. The catheter tube 312 includes two catheter-tube lumens 413 and the optical-fiber stylet 424 disposed in a longitudinal bead of the catheter tube 312 such as between the two catheter-tube lumens 413, as extruded. Optionally, in a same or different longitudinal bead of the catheter tube 312, the PICC 310 can further include the ECG stylet. The bifurcated hub 314 has two hub lumens correspondingly fluidly connected to the two catheter-tube lumens 413. Each extension leg of the two extension legs 316 has an extension-leg lumen fluidly connected to a hub lumen of the two hub lumens. The PICC 310 further includes the extension tube 320 either extending from the bifurcated hub 314 or communicatively coupled to the bifurcated hub 314. When extending from the bifurcated hub 314, the extension tube 320 can be a skived portion of the catheter tube 312 including the optical-fiber stylet 424 and, if present, the ECG stylet, which extension tube 320 can terminate in the plug 322 for establishing an optical connection between the optical-fiber stylet 424 of the PICC 310 and the optical fiber 628 of the relay module 120, as well as any electrical connections. The skived portion of the catheter tube 312 can be disposed in another tube, which, in combination, forms the extension tube 320 terminating in the plug 322 for establishing the foregoing optical and electrical connections.

While the PICC 310 is provided as a particular embodiment of the OSS medical device 110 of the medical system 100 or 200, it should be understood that any of a number of medical devices including catheters such as a CVC can include at least an optical-fiber stylet and, optionally, electrical componentry such as the ECG stylet and the one-or-more electrical leads in support thereof, terminating in a plug for establishing an optical or optical-and-electrical connection between the medical device and the relay module 120.

### Relay Modules

FIG. 6 illustrates a detailed view of the relay module 120 with a primary receptacle 632 for establishing optical or optical-and-electrical connections in accordance with some embodiments. FIG. 7 illustrates establishing a through-barrier optical or optical-and-electrical connection between the OSS medical device 110 and the relay module 120 in accordance with some embodiments. FIG. 9 illustrates the second medical system 200 in use during a patient procedure with the medical barrier 703 in accordance with some embodiments.

As shown, the relay module 120 includes the housing 324, the primary receptacle 632 disposed in the housing 324, one or more secondary receptacles such as secondary receptacle 634 disposed in the housing 324, one or more cables extending from the housing including a primary cable such as the cable 326 extending from the housing 324, and the optical fiber 628 extending through the housing 324 and along the cable 326. Again, the relay module 120 can include one or more electrical leads extending through the housing 324 and along the cable 326 or another cable (e.g., a secondary cable) of the one-or-more cables.

The primary receptacle 632 includes an optical receiver configured to accept insertion of an optical terminal of a plug of the OSS medical device 110 (e.g., the plug 322 of the PICC 310) for establishing an optical connection (e.g., a through-barrier optical connection) between the relay module 120 and the optical-fiber stylet of the OSS medical device 110 (e.g., the optical-fiber stylet 424 of the PICC 310) when the plug is inserted into the primary receptacle 632. The primary receptacle 632 can also include one or more electrical contacts configured to contact an electrical terminal of the plug of the OSS medical device 110 (e.g., a metal piece of the plug 322 of the PICC 310 corresponding to a barrier-piercing element 523 of the plug 322), when present, for establishing an electrical connection between the relay module 120 and the one-or-more electrical leads of the OSS medical device 110 simultaneously with the optical connection in an optical-and-electrical connection (e.g., a through-barrier optical-and-electrical connection) when the plug is inserted into the primary receptacle 632.

The one-or-more secondary receptacles (e.g., the secondary receptacle 634) are configured to accept insertion of a corresponding number of electrical plugs associated with the one-or-more electrical medical devices (e.g., the ECG patches 112, the arm band 114, etc.) and establish a corresponding number of electrical connections (e.g., under-barrier electrical connections) between the relay module 120 and the one-or-more electrical medical devices. For example, the relay module 120 of FIGS. 1 and 2 includes at least three secondary receptacles to accept insertion of three electrical plugs associated with the ECG patch 112a (e.g., a right-arm ECG patch), the ECG patch 112b (e.g., a left-leg ECG patch), and, optionally, the arm band 114 and establish three electrical connections (e.g., under-barrier electrical connections) between the relay module 120 and the foregoing electrical medical devices.

The one-or-more cables include one or more corresponding plugs for establishing one or more connections between the relay module 120 and a remainder of the medical system 100 or 200, which is configured to receive optical and electrical signals from the relay module 120. For example, the primary cable (e.g., the cable 326) of the one-or-more cables includes a primary plug (e.g., the plug 330) for establishing at least an optical connection (e.g., connection b or b″ in FIG. 2) between the relay module 120 and the console 230 (i.e., the console 230 including the integrated optical interrogator 232) for shape sensing with the OSS medical device 110. Optionally, the primary plug is also for establishing an electrical connection (e.g., connection b″ in FIG. 2) between the relay module 120 and the console 230 for at least generating ECG traces with the ECG stylet. Exclusive of the foregoing electrical connection (i.e., that of connection b″ in FIG. 2), the one-or-more cables can include, for example, a secondary cable with a secondary plug for establishing one or more electrical connections (e.g., connection b′ in FIG. 2) between the relay module 120 and the console 230 for generating ECG traces with the ECG patches 112, determining a reference frame with the arm band 114, or both. However, in some embodiments, only the primary cable and plug is needed for establishing all such optical and electrical connections. (See connection b″ in FIG. 2.)

The optical fiber 628 extends from the primary receptacle 632 through the primary cable (e.g., the cable 326) to the primary plug (e.g., the plug 330). The optical fiber 628 is configured to convey the input optical signals from the optical interrogator 140 or 232 to the optical-fiber stylet of the OSS medical device 110 (e.g., the optical-fiber stylet 424 of the PICC 310) and the reflected optical signals from the optical-fiber stylet to the optical interrogator 140 or 232.

As set forth above, the relay module 120 can further include the one-or-more sensors 222 selected from the gyroscope, the accelerometer, and the magnetometer disposed within the housing 324. The one-or-more sensors 222 are configured to provide sensor data for determining a reference plane for shape sensing with the optical-fiber stylet of the OSS medical device 110 (e.g., the optical-fiber stylet 424 of the PICC 310).

The relay module 120 can further include an LED 636 disposed in the housing 324 proximate the primary receptacle 632. The LED 636 is configured to illuminate upon establishing an optical-and-electrical connection (e.g., connection b″ in FIG. 2) or another electrical connection (e.g., connection b′ in FIG. 2). When illuminated, the LED 636 is useful for finding the primary receptacle 632 when the relay module 120 is under the medical barrier 703.

The relay module 120 further includes one or more electrical leads extending from any number of receptacles including the primary receptacle 632 and the one-or-more secondary receptacles, the one-or-more sensors 222, if present, and the LED 636, if present, through any of the one-or-more cables (e.g., the cable 326) and corresponding plugs (e.g., the plug 330) thereof. In addition to any needed electrical power by the ECG stylet, the one-or-more electrical medical devices connected to the secondary receptacles, the one-or-more sensors 222, the LED 636, or the like, the one-or-more electrical leads are configured to convey input electrical signals from the console 130 or 230 to the ECG stylet, the one-or-more electrical medical devices connected to the secondary receptacles, the one-or-more sensors 222, the LED 636, or the like. The one-or-more electrical leads are also configured to convey any output electrical signals from the ECG stylet, the one-or-more electrical medical devices connected to the secondary receptacles, the one-or-more sensors 222, the LED 636, or the like to the console 130 or 230.

The relay module 120 is configured to sit beneath the medical barrier 703 on or alongside a patient P such as on a chest of the patient. As such, the relay module 120 need not require disinfection or sterilization. However, should the relay module 120 require disinfection or sterilization, the relay module 120 can be configured to be amenable to disinfection or sterilization. For example, the housing 324 of the relay module 120 can be non-porous or chemically resistant to oxidants. The relay module 120 can be configured for manual disinfection with a ChloraPrep® product by Becton, Dickinson and Company (Franklin Lakes, NJ), or the relay module 120 can be configured for automatic high-level disinfection or sterilization with vaporized H₂O₂ by way of Trophon® by Nanosonics Inc. (Indianapolis, IN).

While not shown, the housing 324 of the relay module 120 can include a loop extending from the housing 324, a tether point integrated into the housing 324, or a ball-lock-pin receiver integrated into the housing 324 configured for attaching a neck strap to the relay module 120. The loop, the tether point, or the ball-lock-pin receiver enables the relay module 120 to be secured to a neck of the patient P while sitting on the patient's chest. Additionally or alternatively, the housing 324 includes a patient-facing surface (e.g., a back of the relay module 120) configured to be adhered to the patient's chest. The patient-facing surface enables the relay module 120 to be secured to the patient while sitting on or alongside the patient whether or not the relay module 120 is also secured to the patient's neck.

Again, the primary receptacle 632 includes the optical receiver configured to accept insertion of the optical terminal of the plug of the OSS medical device 110 (e.g., the plug 322 of the PICC 310) and form an optical connection when the plug is inserted into the primary receptacle 632. The primary receptacle 632 can also include one or more electrical contacts configured to contact the electrical terminal (e.g., a metal piece of the plug 322 corresponding to the barrier-piercing element 523 of the plug 322) of the plug of the OSS medical device 110, when present, for establishing an electrical connection between the relay module 120 and the one-or-more electrical leads of the OSS medical device 110 when the plug is inserted into the primary receptacle 632. However, with the relay module 120, such optical and electrical connections are formed with the medical barrier 703 between the relay module 120 and the OSS medical device 110. The primary receptacle 632 and the plug of the OSS medical device 110 enable such connections from a sterile field (e.g., above the medical barrier 703 when a sterile drape) including the OSS medical device 110 such as the PICC 310 to a non-sterile field (e.g., beneath the medical barrier 703 when a sterile drape) including the relay module 120.

### Other Embodiments

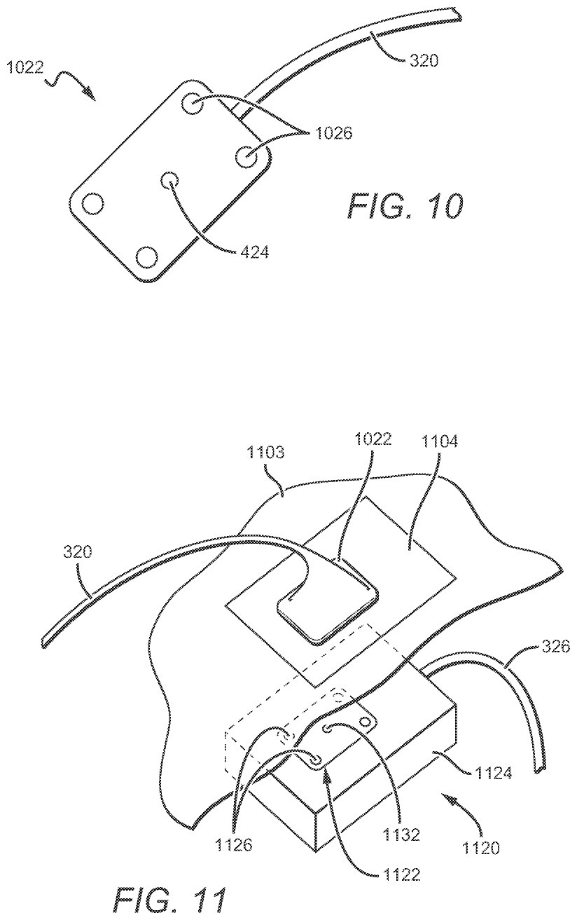

FIG. 10 illustrates an optical connector 1022 of the extension tube 320 of the OSS medical device 110 in accordance with some embodiments. FIG. 11 illustrates an alternative relay module 1120 with an optical connector 1122 for establishing optical connections across a medical barrier 1103 in accordance with some embodiments.

As set forth above, the extension tube 320 can include one or more optical-fiber cores extending from the optical-fiber stylet 424 along a length of the extension tube 320. The one-or-more optical-fibers can extend to an optical terminal in a mating surface of the optical connector 1022.

The optical connector 1022 includes one or more alignment magnets 1026 disposed in the mating surface of the optical connector 1022 around the optical terminal or an end portion of the optical-fiber stylet 424.

As set forth above, the relay module 120 can be configured to relay optical signals to a receiver thereof such as the console 230 of the medical system 200. When the relay module 1120 is so configured, the relay module 1120 includes one or more optical-fiber cores within a housing 1124 of the relay module 1120 and the optical connector 1122. It should be understood the relay module 1120 can be further configured to relay electrical signals from the one-or-more electrical devices (e.g., the ECG patch 112a, the ECG patch 112b, the arm band 114, etc.) to the console 230 of the medical system 200 like the relay module 120.

The optical connector 1122 includes one or more alignment magnets 1126 disposed in a mating surface of the optical connector 1122 around an optical receiver 1132.

The optical connector 1022 and the optical connector 1122 are configured to mate across a transparent window 1104 of the medical barrier 1103 (e.g., a drape) and establish an optical connection between the optical terminal of the optical connector 1022 in a sterile field and the optical receiver of the optical connector 1122 in a non-sterile field.

A shape of each connector of the optical connector 1022 and the optical connector 1122 can be configured to enforce a particular orientation of the optical connector 1022 and the optical connector 1122 when mated across the transparent window 1104 of the medical barrier 1103. For example, each connector of the optical connector 1022 and the optical connector 1122 shown in FIG. 11 is rectangular or longer than it is wide, thereby enforcing two of the four most reasonable orientations for rectangular connectors.

Magnetic poles of the one-or-more alignment magnets 1026 and 1126 of each connector of the optical connector 1022 and the optical connector 1122 can additionally or alternatively be configured to enforce a particular orientation of the optical connector 1022 and the optical connector 1122 when mated across the transparent window 1104 of the medical barrier 1103. For example, a first side of the optical connector 1022 can include a first pair of the alignment magnets 1026 having a same magnetic pole orientation (e.g., N). A second side of the optical connector 1022 can include a second pair of the alignment magnets 1026 having a same magnetic pole orientation (e.g., S) but different than the first side of the extension-tube connector. The optical connector 1122 can be likewise configured such that similar sides of the optical connector 1022 and the optical connector 1122 repel each other when brought close to each other and dissimilar sides of the optical connector 1022 and the optical connector 1122 attract each other when brought close to each other. In this way, two of the four most reasonable orientations of, for example, square-shaped connectors can be enforced. However, if the optical connector 1022 and the optical connector 1122 are rectangular as shown in FIG. 11, both the shape and the magnetic poles configured as in the example can enforce a single orientation.

Notwithstanding the foregoing, a shape of each connector of the optical connector 1022 and the optical connector 1122 can be rotationally symmetric. Such a configuration allows a number of rotationally equivalent orientations of the optical connector 1022 and the optical connector 1122 when mated across the transparent window 1104 of the medical barrier 1103. For example, all the magnetic poles of the one-or-more alignment magnets 1026 of the optical connector 1022 can be of a same magnetic pole orientation but opposite all the magnetic poles of the one-or-more alignment magnets 1126 of the optical connector 1122 to complement all the magnetic poles of the one-or-more alignment magnets 1126 of the optical connector 1122. Indeed, such a configuration allows a number of rotationally equivalent orientations of the optical connector 1022 and the optical connector 1122 when mated across the transparent window 1104 of the medical barrier 1103.

### Methods

FIG. 9 illustrates the second medical system 200 in use during a patient procedure with the medical barrier 703 in accordance with some embodiments.

A method of the medical system 100 or 200 can include a method of use during a medical procedure. For example, a method of using the medical system 200 during such a medical procedure includes a relay-module placing step, an electrical-plug inserting step, a medical-drape placing step, and a barrier-piercing-plug inserting step.

The relay-module placing step includes placing the relay module 120 on a surface (e.g., a relatively flat surface such as a chest of the patient P, a table beside the patient P, etc.), which establishes a reference plane for shape sensing with the OSS medical device 110. Prior to the relay-module placing step, the method can further include a disinfecting or sterilizing step of disinfecting or sterilizing the relay module 120 before placing the relay module 120 on or alongside the patient P.

The electrical-plug inserting step includes inserting one or more electrical plugs corresponding to one or more electrical medical devices into a corresponding number of secondary receptacles (e.g., the secondary receptacle 634) of the relay module 120. The electrical-plug inserting step establishes one or more under-barrier electrical connections—or soon-to-be one or more under-barrier electrical connections—between the one-or-more electrical devices and the relay module 120.

The medical-drape placing step includes placing the medical barrier 703 over the relay module 120, as well as any of the one-or-more electrical medical devices if already connected to the relay module 120. The medical-drape placing step establishes a sterile field over the relay module 120 and any of the one-or-more electrical devices connected thereto.

The barrier-piercing-plug inserting step includes inserting the barrier-piercing plug 322 of the OSS medical device (e.g., the PICC 310) through the medical barrier 703 and into the primary receptacle 632 of the relay module 120. The barrier-piercing-plug inserting step establishes a through-barrier optical connection or a through-barrier optical-and-electrical connection between the OSS medical device 110 in the sterile field and the relay module 120 in a non-sterile field.

The method further can further include an identifying step, a palpating step, or both. The identifying step includes identifying the LED 636 disposed in the housing 324 of the relay module 120 proximate the primary receptacle 632. The identifying step occurs after the establishing step set forth below, which illuminates the LED 636. The palpating step includes palpating the primary receptacle 632 under the medical barrier 703. Both the identifying step and the palpating step occur before the barrier-piercing-plug inserting step.

The method can further include an establishing step of establishing one or more connections between the relay module 120 and a remainder of the medical system 200 (e.g., the console 230) for relaying optical and electrical signals from the relay module 120. The one-or-more connections are selected from an optical connection, an optical-and-electrical connection, and an electrical connection. At least the optical-and-electrical connection (e.g., connection b″ in FIG. 2) or the electrical connection as another electrical connection (e.g., connection b′ in FIG. 2) between the relay module 120 and the console 230 are needed to provide power to the LED 636 for illumination thereof for the identifying step.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A method of a medical system, comprising:
placing a relay module on a surface;
inserting one or more electrical plugs corresponding to one or more electrical medical devices into a corresponding number of secondary receptacles of the relay module, thereby establishing one or more under-barrier electrical connections between the one or more electrical devices and the relay module;
placing a medical barrier over the relay module, thereby establishing a sterile field over the relay module; and
inserting a barrier-piercing plug of an optical shape-sensing (“OSS”) medical device through the medical barrier and into a primary receptacle of the relay module, thereby establishing a through-barrier optical connection or a through-barrier optical-and-electrical connection between the OSS medical device in the sterile field and the relay module in a non-sterile field.

2. The method of claim 1, further comprising establishing one or more relay connections between the relay module and a remainder of the medical system for relaying optical and electrical signals from the relay module, the one or more relay connections selected from a relay optical connection, a relay optical-and-electrical connection, and a relay electrical connection, provided at least the relay optical connection or the relay optical-and-electrical connection is selected.

3. The method of claim 1, wherein the relay module includes one or more sensors selected from a gyroscope, an accelerometer, and a magnetometer disposed within a housing of the relay module, the one or more sensors configured to provide sensor data for determining a reference plane for shape sensing with the OSS medical device.

4. The method of claim 3, wherein placing the relay module on the surface includes placing the relay module on a table beside a patient, thereby establishing the reference plane for shape sensing with the OSS medical device.

5. The method of claim 3, wherein placing the relay module on the surface includes placing the relay module on a chest of a patient, thereby establishing the reference plane for shape sensing with the OSS medical device.

6. The method of claim 5, wherein placing the relay module on the chest of the patient includes adhering a patient-facing surface of the housing of the relay module to the patient, thereby securing the relay module to the patient.

7. The method of claim 1, wherein the relay module is amenable to high-level disinfection or sterilization.

8. The method of claim 7, further comprising disinfecting the relay module before placing the relay module on a chest of a patient as the surface, thereby enabling a reference plane to be established for shape sensing with the OSS medical device.

9. The method of claim 1, further comprising identifying an illuminated light-emitting diode (“LED”) disposed in a housing of the relay module proximate the primary receptacle before the inserting of the barrier-piercing plug of the OSS medical device into the primary receptacle of the relay module.

10. The method of claim 9, wherein the LED disposed in the housing of the relay module is configured to illuminate upon establishing the one or more under-barrier electrical connections between the one or more electrical devices and the relay module for finding the primary receptacle of the relay module under the medical barrier.

11. The method of claim 1, further comprising palpating the primary receptacle under the medical barrier before the inserting of the barrier-piercing plug of the OSS medical device into the primary receptacle of the relay module.
